(19) United States
(12) Patent Application Publication
(10) Pub. No.: US 2023/0248143 A1
(43) Pub. Date: Aug. 10, 2023

(54) Artificial Intelligence-Enabled Furniture System with Air Purifier

(71) Applicant: Guan Leng LIM, Singapore (SG)

(72) Inventor: Guan Leng LIM, Singapore (SG)

(21) Appl. No.: 17/668,102

(22) Filed: Feb. 9, 2022

(51) Int. Cl. A47B 83/04; A61L 9/20; A47B 81/06

(52) U.S. Cl. CPC A47B 83/04; A61L 9/20; A47B 81/06; A61L 2209/14; A47B 2200/008; A47B 2220/0097

(57) ABSTRACT

The present disclosure discloses an artificial intelligence-enabled electronic furniture system with air purifier. The system comprises an assembly including a table top with a plurality of near field communication (NFC) devices placed at a predetermined position for communication between two or more devices. The system further comprises a speaker enclosure associated with the table top through a plurality of first connecting means present at the bottom of the speaker enclosure. An air purifier compartment is attached to the speaker enclosure through a plurality of second connecting means. Further, the air purifier compartment comprises a UV filter configured to inactivate airborne pathogens and microorganisms from the air. A motor mount includes an electric motor with a fan. A plurality of legs attached to the assembly and are used to support the constructed table. The system further includes a platform for controlling the air purifier using the near field communication (NFC) device.

### BACKGROUND

### Field of Invention

The present invention relates to multi-function furniture system, and more particularly, to an artificial intelligence-enabled furniture system with air purifier.

### Description of Related Art

Air pollution is a known fact in our workspaces and homes as a result of living in an urbanized city. To alleviate worry about air contaminants, many indoor places are equipped with air purifiers. Another pressing concern for city dwellers is the reducing living areas, with tiny dwellings becoming the norm. There are a variety of air purifiers on the market in various sizes, but with the limited space in smart compact houses, it might be difficult to fit many appliances.

Tables, chairs, beds, desks, dressers, wardrobes, and other types of cupboards are examples of bi- or three-dimensional movable, fixed, or built-in furniture. These items are typically kept in a house or other structure to make it appropriate for living or working in.

The market produced several types of air purifying equipment to make the air clean and free of hazardous chemicals because there is a requirement for a healthy atmosphere in living and working areas.

Further, on a regular basis, a user may trip over a big air purifier in a cozy bedroom and desire for more space for speakers to play music to help them sleep. Traditional purifier-equipped furniture is not multifunctional, big, takes up more room, and consumes more energy.

There is currently a gap in the market for an AI-enabled furniture system with air purifier that will be ideal for providing tiny homes with clean air and convenience of a multifunctional device all in one.

### SUMMARY

Embodiments in accordance with the present invention provide an artificial intelligence-enabled electronic furniture system. The system also includes a table-top with a plurality of near field communication (NFC) devices placed at a predetermined position for communication between two or more devices. The system also includes a speaker enclosure associated with the table-top through a plurality of first connecting means present at the bottom of the speaker enclosure. The speaker enclosure includes a plurality of speakers screwed to the walls of the speaker enclosure. Further, the speaker enclosure comprises printed circuit boards (PCBs), a battery, a wireless charging module, and other electronic circuitry placed in the speaker enclosure. The battery is configured to power the speakers, and work as a power bank for a mobile device charging. The system further includes an air purifier compartment attached to the speaker enclosure through a plurality of second connecting means. The air purifier compartment is configured to hold an air purifier that is set up to remove air pollutants in indoor spaces and provide clean air through a plurality of air vents. Furthermore, the air purifier compartment comprises a UV filter configured to inactivate airborne pathogens and microorganisms from the air. The air purifier compartment also comprises a motor mount including an electric motor with a fan. The system also includes a plurality of legs attached to the assembly and are used to support the constructed table. The system also includes a platform for controlling the air purifier using the near field communication (NFC) device.

Embodiments in accordance with the present invention provide a process of making an artificial intelligence-enabled electronic furniture system. The process includes creating a table-top with a plurality of near field communication (NFC) devices placed at a predetermined position for communication between two or more devices. The process also includes arranging a speaker enclosure associated with the table-top through a plurality of first connecting means present at the bottom of the speaker enclosure. The speaker enclosure includes a plurality of speakers screwed to the walls of the speaker enclosure. Further, the speaker enclosure comprises printed circuit boards (PCBs), a battery, a wireless charging module, and other electronic circuitry placed in the speaker enclosure. The battery is configured to power the speakers, and work as a power bank for a mobile device charging. The process also includes attaching an air purifier compartment to the speaker enclosure through a plurality of second connecting means. The air purifier compartment is configured to hold an air purifier that is set up to remove air pollutants in indoor spaces and provide clean air through a plurality of air vents. The process also includes inactivating airborne pathogens and microorganisms from the air through a UV filter. The process also includes mounting an electric motor with a fan in a motor mount. The process also includes attaching a plurality of legs to the assembly to support the constructed table. The process also includes controlling the air purifier through a platform using the near field communication (NFC) device.

These and other advantages will be apparent from the present application of the embodiments described herein.

The preceding is a simplified summary to provide an understanding of some embodiments of the present invention. This summary is neither an extensive nor exhaustive overview of the present invention and its various embodiments. The summary presents selected concepts of the embodiments of the present invention in a simplified form as an introduction to the more detailed description presented below. As will be appreciated, other embodiments of the present invention are possible utilizing, alone or in combination, one or more of the features set forth above or described in detail below.

### BRIEF DESCRIPTION OF THE DRAWINGS

The above and still further features and advantages of embodiments of the present invention will become apparent upon consideration of the following detailed description of embodiments thereof, especially when taken in conjunction with the accompanying drawings, and wherein:

FIG. 1A illustrates a perspective view of an artificial intelligence-enabled furniture system with an air purifier, according to embodiments of the present invention disclosed herein;

FIG. 1B illustrates a front view of the artificial intelligence-enabled furniture system with an air purifier, according to embodiments of the present invention disclosed herein;

FIG. 1C illustrates a sectional view of a speaker enclosure of the artificial intelligence-enabled furniture system with an air purifier, according to embodiments of the present invention disclosed herein;

FIG. 1D illustrates a cross-sectional view of the artificial intelligence-enabled furniture system with an air purifier, according to embodiments of the present invention disclosed herein;

FIG. 1E illustrates an exploded view an artificial intelligence-enabled furniture system with an air purifier, according to embodiments of the present invention disclosed herein;

FIG. 1F illustrates an expanded view an artificial intelligence-enabled furniture system with an air purifier, according to embodiments of the present invention disclosed herein; and

FIG. 2 illustrates a flowchart of a process of making an artificial intelligence-enabled electronic furniture system with an air purifier, according to embodiments of the present invention disclosed herein.

The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include”, “including”, and “includes” mean including but not limited to. To facilitate understanding, like reference numerals have been used, where possible, to designate like elements common to the figures. Optional portions of the figures may be illustrated using dashed or dotted lines, unless the context of usage indicates otherwise.

### DETAILED DESCRIPTION

The following description includes the preferred best mode of one embodiment of the present invention. It will be clear from this description of the invention that the invention is not limited to these illustrated embodiments, but the invention also includes a variety of modifications and embodiments thereto. Therefore, the present description should be seen as illustrative and not limiting. While the invention is susceptible to various modifications and alternative constructions, it should be understood, that there is no intention to limit the invention to the specific form disclosed, but, on the contrary, the invention is to cover all modifications, alternative constructions, and equivalents falling within the spirit and scope of the invention as defined in the claims.

In any embodiment described herein, the open-ended terms “comprising,” “comprises,” and the like (which are synonymous with “including,” “having” and “characterized by”) may be replaced by the respective partially closed phrases “consisting essentially of,” consists essentially of,” and the like or the respective closed phrases “consisting of,” “consists of, the like.

As used herein, the singular forms “a”, “an”, and “the” designate both the singular and the plural, unless expressly stated to designate the singular only.

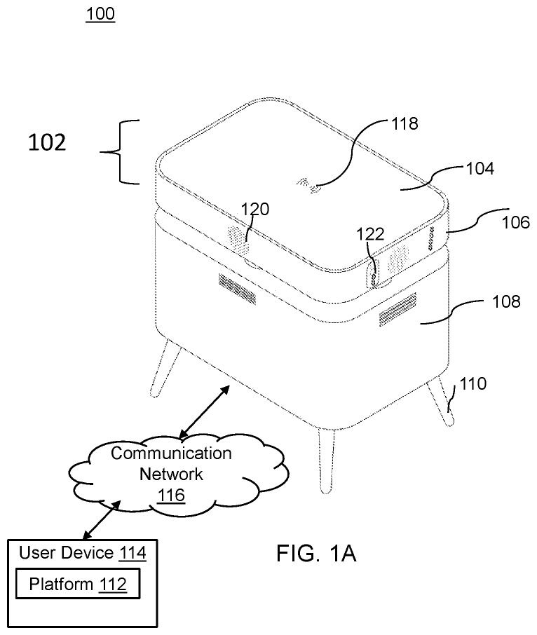

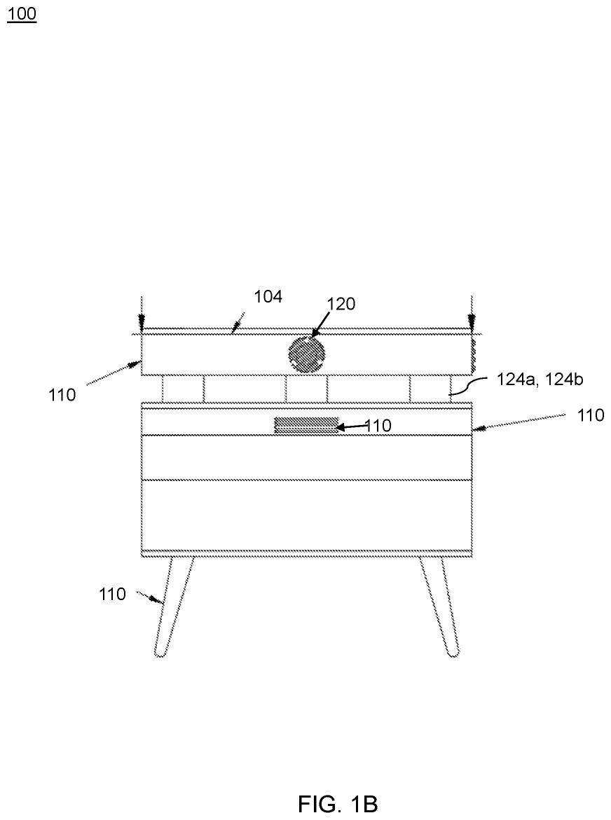

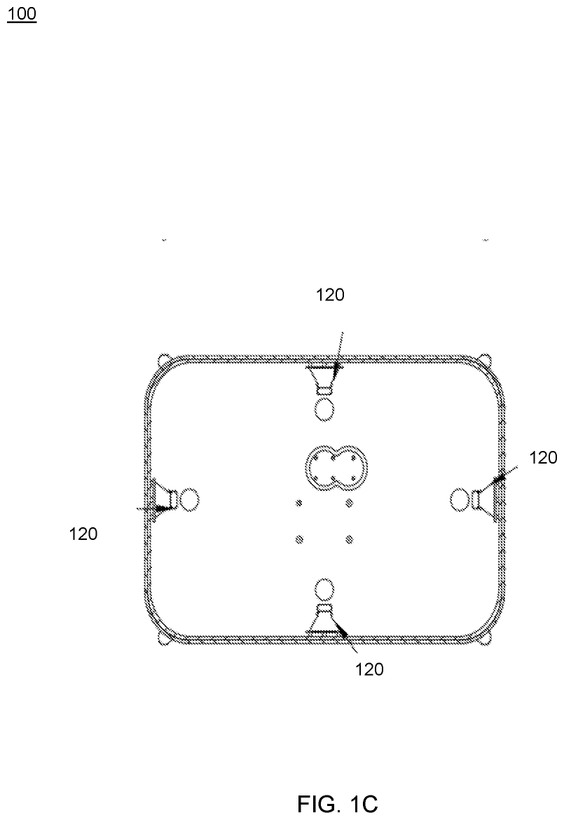

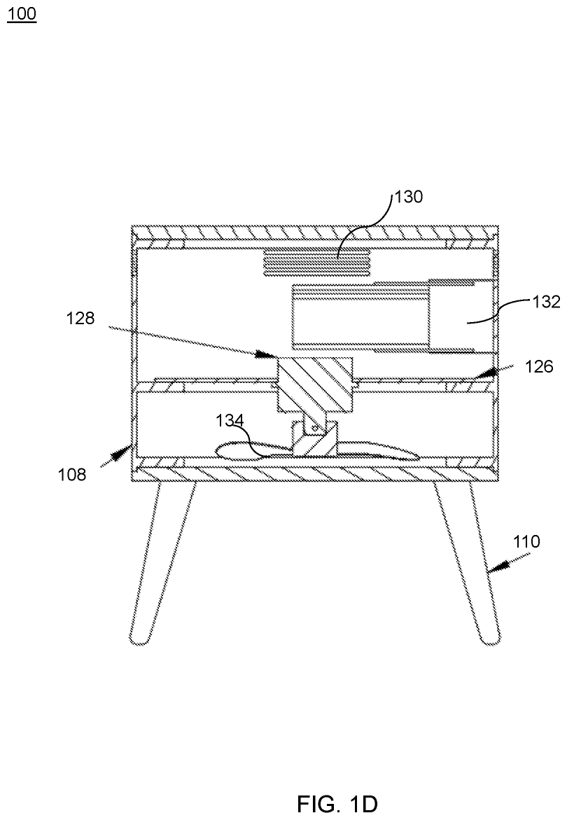

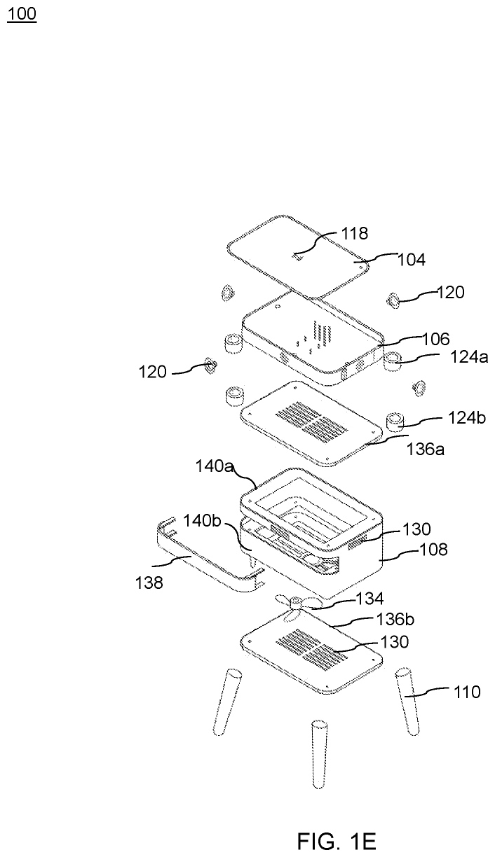

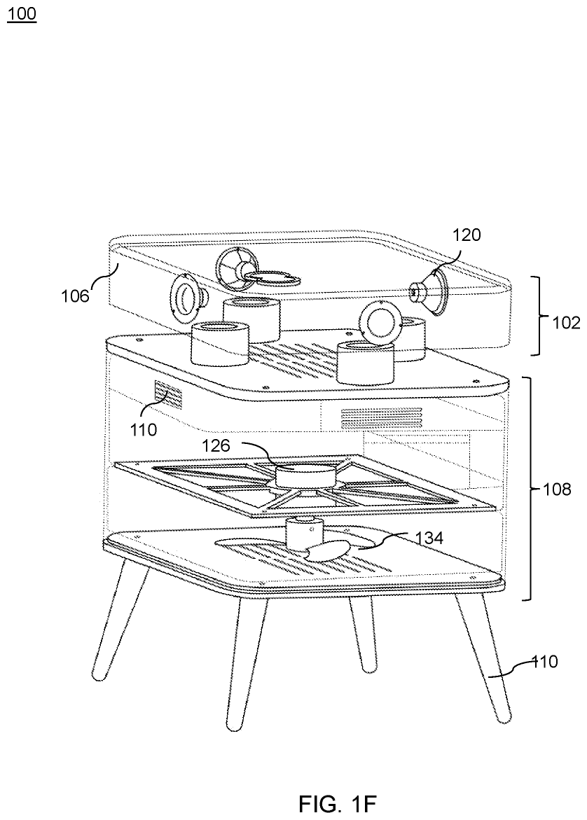

FIG. 1A illustrates a perspective view of an artificial intelligence-enabled furniture system 100 with an air purifier, according to embodiments of the present invention. The system 100 comprises a first assembly 102 comprising a table-top 104 and a speaker enclosure 106, an air purifier compartment 108, a plurality of legs 110, and a platform 112 installed on a user device 114, according to embodiments of the present invention.

The first assembly 102 comprising the table-top 104 with a plurality of near field communication (NFC) devices 118 placed at a predetermined position for communication between two or more devices. The near field communication (NFC) device 118 may comprise a Bluetooth device, a wireless configuration device, and so forth. Embodiments of the present invention are intended to include or otherwise cover any type of the NFC device 118 including known, related art, and/or later developed technologies. In preferred embodiments of the present invention, the wireless configuration device may be located in the middle of the table-top 104. Further the table-top 104 may be attached to the speaker enclosure 106 through an attaching means.

According to embodiments of the present invention, the table-top 104, the speaker enclosure 106, the air purifier compartment 108 may be made of a material comprising, wood, plastic, any metal, and so forth. In preferred embodiment of the present invention, the table-top 104, the speaker enclosure 106, the air purifier compartment 108 may be made of an acoustic fabric with wood. Furthermore, the table-top 104, the speaker enclosure 106, the air purifier compartment 106 may be of any shape or size.

According to embodiments of the present invention, the attaching mechanism maybe an adhesive. In another embodiment of the present invention, the table-top 104 may be simply placed over the speaker enclosure 106. The speaker enclosure 106 may comprise a plurality of charging ports 122 configured to charge mobile devices or user device 114.

In an embodiment the speaker enclosure 106 may comprise printed circuit boards (PCBs), a battery, a wireless charging module, and other electronic circuitry placed in the speaker enclosure 106. The battery may be configured to power the speakers, and work as a power bank for a mobile device charging.

In a preferred embodiment of the present invention, the platform 112 may be installed on the user device 114 for controlling the air purifier using the near field communication (NFC) device 118. The user device 114 can be connected to the system 100 through a communication network to control the fan 134 speed and turn on/off the electric motor 128 positioned in the motor mount 126.

In preferred embodiments of the present invention, the table-top 104, speaker enclosure 106, and air purifier compartment 108 all have a rectangle shape with curved edges. In other embodiments of the present invention, the table-top, speaker enclosure, and air purifier compartment 106 may be rectangular, circular, oval, and so forth.

The air purifier compartment 108 may be coupled to the speaker enclosure 106 using first and second connection means 124a, 124b that are placed into each other, as seen in FIG. 1B. In preferred embodiments of the present invention, four legs 110 may be attached to the bottom of the air purifier compartment 108. The speaker enclosure 106 may comprise a plurality of first connecting means present at the bottom of the speaker enclosure 106, as seen particularly in FIG. 1B. The air purifier compartment 108 has a plurality of air vents for air circulation on the top, bottom, and sides of the compartment 108.

FIG. 1C illustrates a sectional view of a speaker enclosure 106 of the artificial intelligence-enabled furniture system 100 with an air purifier, according to embodiments of the present invention. The speaker enclosure 106 may include a plurality of speakers 120 screwed to the walls of the speaker enclosure 106. One speaker 120 may be mounted to each wall opposing each other of the speaker enclosure 106 in preferred embodiments of the present invention.

FIG. 1D illustrates a cross-sectional view of the artificial intelligence-enabled furniture system 100 with the air purifier, according to embodiments of the present invention. The air purifier compartment 108 may be attached to the speaker enclosure 106 through the plurality of second connecting means 124b. The air purifier compartment 106 may be configured to hold the air purifier that is set up to remove air pollutants in indoor spaces and provide clean air through a plurality of air vents 130. Further, the air purifier compartment 108 may comprise a UV filter 132 that may be configured to inactivate airborne pathogens and microorganisms from the air. The UV filter 132 may be replaceable.

According to an embodiment of the present invention, the air purifier compartment 106 is made up of two enclosures 140a, 140b that are linked at a predetermined height, with a third enclosure covering the space on three sides. The two enclosures 140a, 140b are rectangular in shape with curving edges. one of the two enclosures 140a put on top of the air purifier compartment 106 has an air vent 130 on each side. In a preferred embodiment of the present invention, one of the two enclosures 140a on top is thinner than the one at the bottom 140b.

In an embodiment of the present invention, a motor mount 126 may include an electric motor 128 with a fan 134, referring to FIGS. 1D and 1E. The fan 134 may circulate the air evenly and push the clean air out of the air vents 130 by turning with the help of the electric motor 128.

FIG. 1E illustrates an exploded view an artificial intelligence-enabled furniture system 100 with the air purifier, according to embodiments of the present invention. The assembly 102 may be formed by placing the table-top 104 on top of the speaker enclosure 106, as seen in FIG. 1F. The speaker enclosure 106 may then be connected to the air purifier compartment 108 using the first and second connecting means 124a and 124b, respectively. The plurality of legs 110 may also be added to the assembly 102 and utilized to support the assembled table.

In an embodiment of the present invention, the air purifier compartment 106 may comprise a closing enclosure 138 detachably attached to the open side of the air purifier compartment 106.

At each side of the speaker enclosure 106, one of the plurality of first connection means 124a may be located in the middle. Further, one of the plurality of second connecting means 124b may be set in the middle of each side of the purifier compartment's top lid 136a. Each of the table-top's first connecting means 124a may be fitted into the second connecting means 124b linked to the air purifier compartment's top lid 136a.

The top lid 136a and the bottom lid 136b may fitted to either side of the air purifier compartment, as seen in FIGS. 1E and 1F. Further, the top lid 136a of the air purifier compartment 106 may comprise one of the pluralities of air vents 130 located in middle.

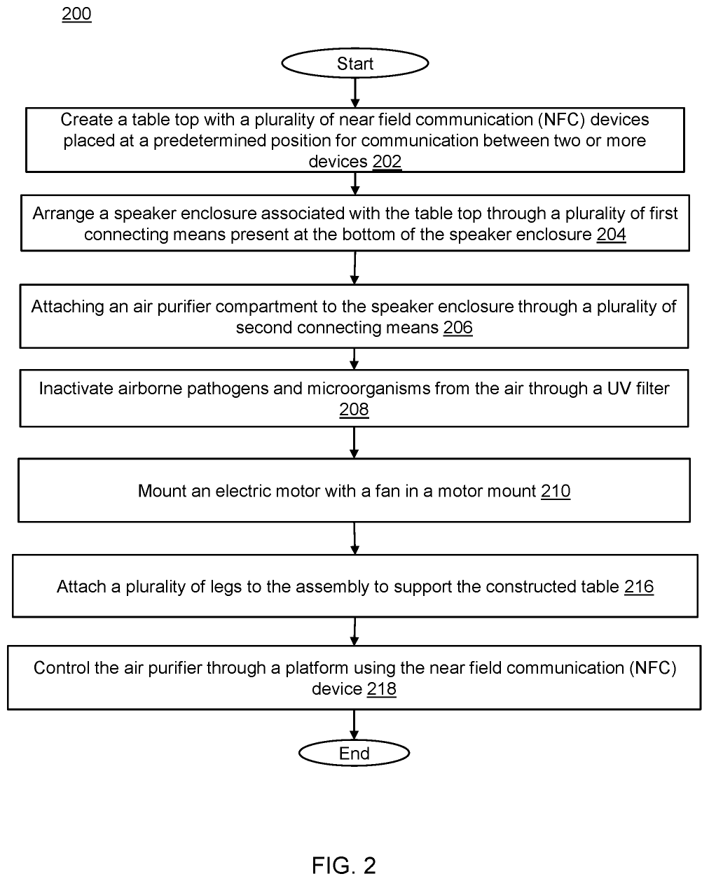

FIG. 2 illustrates a flowchart of a process 200 of making an artificial intelligence-enabled electronic furniture system, according to embodiments of the present invention.

At 202, the table-top 104 may be created by embedding the plurality of near field communication (NFC) devices 118 placed at a predetermined position for communication between two or more devices.

At 204, the speaker enclosure may be associated with the table-top 104 through the plurality of first connecting means 124a present at the bottom of the speaker enclosure 106. The speaker enclosure 106 may include the plurality of speakers 120 screwed to the walls of the speaker enclosure 106. In an embodiment of the present invention, the speaker enclosure 06 may comprise printed circuit boards (PCBs), a battery, a wireless charging module, and other electronic circuitry placed in the speaker enclosure. Further, the battery may be configured to power the speakers, and work as a power bank for a mobile device charging.

At 206, the air purifier compartment 108 may be attached to the speaker enclosure 106 through the plurality of second connecting means 124b. The air purifier compartment 108 may be configured to hold the air purifier that is set up to remove air pollutants in indoor spaces and provide clean air through the plurality of air vents 130.

At 208, airborne pathogens and microorganisms may be inactivated or removed from the air through the UV filter 132 associated with the air purifier compartment 108.

At 210, the electric motor 128 with the fan 134 may be mounted in the motor mount 128 or motor holder.

At 212, the plurality of legs 110 may be attached to the assembly 102 to support the constructed table.

At 214, the air purifier intact in the air purifier compartment 108 may be controlled through the platform 112 using the near field communication (NFC) device 112.

While the invention has been described in connection with what is presently considered to be the most practical and various embodiments, it is to be understood that the invention is not to be limited to the disclosed embodiments, but on the contrary, is intended to cover various modifications and equivalent arrangements included within the spirit and scope of the appended claims.

This written description uses examples to disclose the invention, including the best mode, and also to enable any person skilled in the art to practice the invention, including making and using any devices or systems and performing any incorporated process. The patentable scope the invention is defined in the claims, and may include other examples that occur to those skilled in the art. Such other examples are intended to be within the scope of the claims if they have structural elements that do not differ from the literal language of the claims, or if they include equivalent structural elements within substantial differences from the literal languages of the claims.

What is claimed:

1. An artificial intelligence-enabled furniture system with an air purifier, the system comprising:
an assembly comprising:
a table-top with a plurality of near field communication (NFC) devices placed at a predetermined position for communication between two or more devices;
a speaker enclosure associated with the table-top through a plurality of first connecting means present at the bottom of the speaker enclosure, the speaker enclosure includes a plurality of speakers screwed to the walls of the speaker enclosure, wherein the speaker enclosure comprises:
printed circuit boards (PCBs), a battery, a wireless charging module, and other electronic circuitry placed in the speaker enclosure, wherein the battery is configured to power the speakers, and work as a power bank for a mobile device charging.
an air purifier compartment attached to the speaker enclosure through a plurality of second connecting means, the air purifier compartment is configured to hold the air purifier that is set up to remove air pollutants in indoor spaces and provide clean air through a plurality of air vents, wherein the air purifier compartment comprises:
a UV filter configured to inactivate airborne pathogens and microorganisms from the air; and
a motor mount includes an electric motor with a fan.
a plurality of legs attached to the assembly and are used to support the constructed table; and
a platform installed on a user device for controlling the air purifier using the near field communication (NFC) device.

2. The system of claim 1, wherein at each side of the speaker enclosure, one of the pluralities of first connection means is located in the middle.

3. The system of claim 1, wherein a top lid and a bottom lid are fitted to either side of the air purifier compartment.

4. The system of claim 3, wherein one of the pluralities of second connecting means is set in the middle of each side of the purifier compartment's top lid.

5. The system of claim 1, wherein each of the table-top's first connecting means is fitted into the second connecting means linked to the air purifier compartment's top lid.

6. The system of claim 1, wherein the table-top, speaker enclosure, and air purifier compartment all have a rectangle shape with curved edges.

7. The system of claim 1, wherein the air purifier compartment is made up of two enclosures that are linked at a predetermined height, with a third enclosure covering the space on three sides.

8. The system of claim 7, wherein the two enclosures are rectangular in shape with curving edges.

9. The system of claim 7, wherein one of the two enclosures put on top of the air purifier compartment has an air vent on each side.

10. The system of claim 7, wherein one of the two enclosures on top is thinner than the one at the bottom.

11. The system of claim 1, wherein the top lid of the air purifier compartment comprises one of the pluralities of air vents located in middle.

12. The system of claim 1, wherein the table-top is joined to the speaker enclosure through an attaching mechanism.

13. The system of claim 1, wherein the air purifier compartment has a number of air vents for air circulation on the top, bottom, and sides of the compartment.

14. The system of claim 1, wherein the UV filter is replaceable.

15. The system of claim 1, wherein the speaker enclosure comprises a plurality of charging ports configured to charge mobile devices.

16. The system of claim 1, wherein the near field communication (NFC) device comprises a Bluetooth device, a wireless configuration device, and any foregoing combination.

17. The system of claim 1, wherein the air purifier compartment comprises a closing enclosure detachably attached to the open side of the air purifier compartment.

18. The system of claim 1, wherein the table-top, the speaker enclosure, the air purifier compartment is made of a material comprising, wood, plastic, any metal or any foregoing combination.

19. A process of making an artificial intelligence-enabled electronic furniture system with an air purifier, the process comprising:
creating a table top with a plurality of near field communication (NFC) devices placed at a predetermined position for communication between two or more devices;
arranging a speaker enclosure associated with the table-top through a plurality of first connecting means present at the bottom of the speaker enclosure, the speaker enclosure includes a plurality of speakers screwed to the walls of the speaker enclosure, wherein the speaker enclosure comprises:
printed circuit boards (PCBs), a battery, a wireless charging module, and other electronic circuitry placed in the speaker enclosure, wherein the battery is configured to power the speakers, and work as a power bank for a mobile device charging;
attaching an air purifier compartment to the speaker enclosure through a plurality of second connecting means, the air purifier compartment is configured to hold the air purifier that is set up to remove air pollutants in indoor spaces and provide clean air through a plurality of air vents;
inactivating airborne pathogens and microorganisms from the air through a UV filter;
mounting an electric motor with a fan in a motor mount;
attaching a plurality of legs to the assembly to support the constructed table; and
controlling the air purifier through a platform using the near field communication (NFC) device.

20. The process of claim 19, wherein the table top, the speaker enclosure, the air purifier compartment is of any shape or size.
